(19) United States
(12) Patent Application Publication
(10) Pub. No.: US 2025/0387175 A1
(43) Pub. Date: Dec. 25, 2025

(54) CONTEXT-AWARENESS SYSTEMS AND METHODS FOR A COMPUTER-ASSISTED SURGICAL SYSTEM

(71) Applicant: Intuitive Surgical Operations, Inc., Sunnyvale, CA (US)

(72) Inventor: Liron Leist, San Jose, CA (US)

(21) Appl. No.: 19/229,753

(22) Filed: Jun. 5, 2025

Related U.S. Application Data
(63) Continuation of application No. 17/055,916, filed on Nov. 16, 2020.
(60) Provisional application No. 62/677,797, filed on May 30, 2018.

(51) Int. Cl. A61B 34/00; A61B 34/10; A61B 34/37; A61B 90/00; G16H 20/40; G16H 40/67

(52) U.S. Cl. CPC A61B 34/25; A61B 34/10; A61B 34/37; A61B 34/70; A61B 90/37; G16H 20/40; G16H 40/67; A61B 2034/254; A61B 2034/256; A61B 2034/258

(57) ABSTRACT

A context-awareness system, which is communicatively coupled to a computer-assisted surgical system during a surgical session in which the computer-assisted surgical system performs one or more operations with respect to a patient, determines that a user device is communicatively paired with the computer-assisted surgical system during the surgical session, identifies a user role associated with the user device, accesses surgical session data generated during the surgical session and based on the one or more operations performed by the computer-assisted surgical system, detects, based on the surgical session data, an event that occurs with respect to the computer-assisted surgical system during the surgical session, identifies, based on the detected event, contextual information associated with the event and that is specific to the user role associated with the user device, and transmits, to the user device, a command for the user device to present the contextual information associated with the event.

### RELATED APPLICATIONS

The present application is a continuation of U.S. patent application Ser. No. 17/055,916, filed Nov. 16, 2020, which is a U.S. National Stage Application under 35 U.S.C. § 371 of International Application No. PCT/US2019/035847, filed Jun. 6, 2019, which claims priority to U.S. Provisional Patent Application No. 62/677,797, filed May 30, 2018, each of which is hereby incorporated by reference in its entirety.

### BACKGROUND INFORMATION

During a surgical procedure that utilizes a computer-assisted surgical system, such as a teleoperated surgical system and/or a surgical system that utilizes robotic technology, a surgical team may coordinate and work together to safely and effectively perform a variety of different tasks. For example, a surgical team that includes a surgeon, one or more nurses, one or more technicians or assistants, and an anesthesiologist may prepare an operating room, set up equipment within the operating room, configure the computer-assisted surgical system, interact with various technical aspects of the equipment and/or computer-assisted surgical system, perform surgical operations on the patient, monitor patient sedation and vital signs, and clean up after the procedure is completed. Each surgical team member may have specific duties that he or she is specifically trained to perform in connection with each of these tasks.

However, coordinating the performance of these tasks by the various different surgical members during a surgical procedure can be challenging, particularly when the surgical team members are not sufficiently familiar with preferences or capabilities of one another or are located in different locations (e.g., when a surgeon using a teleoperated surgical system is located remotely from the patient). Moreover, some surgical team members may not be aware of events that occur during the surgical procedure, such as events that occur out of the view of a particular surgical team member.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary computer-assisted surgical system according to principles described herein.

FIG. 2 illustrates an exemplary manipulating system included within the computer-assisted surgical system of FIG. 1 according to principles described herein.

FIG. 3 illustrates an exemplary manipulator arm included within the manipulating system of FIG. 2 according to principles described herein.

FIG. 4 illustrates an exemplary user control system included within the computer-assisted surgical system of FIG. 1 according to principles described herein.

FIG. 5 illustrates an exemplary stereoscopic endoscope located at an exemplary surgical area associated with a patient according to principles described herein.

FIG. 6 illustrates an exemplary context-awareness system according to principles described herein.

FIG. 7 illustrates an exemplary implementation of the context-awareness system illustrated in FIG. 6 according to principles described herein.

FIG. 8 illustrates an exemplary association table according to principles described herein.

FIGS. 9-10 illustrate exemplary manners in which an event may be detected based on surgical session data according to principles described herein.

FIG. 11 illustrates an exemplary contextual information table according to principles described herein.

FIG. 12 illustrates an exemplary context-awareness method according to principles described herein.

FIG. 13 illustrates an exemplary computing system according to principles described herein.

### DETAILED DESCRIPTION

Context-awareness systems and methods for a computer-assisted surgical system are disclosed herein. As will be described below in more detail, an exemplary context-awareness system may be communicatively coupled to a computer-assisted surgical system during a surgical session in which the computer-assisted surgical system performs one or more operations with respect to a patient. In this configuration, the context-awareness system may determine that a user device (e.g., a smartphone, a tablet computer, or any other computing device) is communicatively paired with the computer-assisted surgical system during the surgical session and identify a user role associated with the user device. The context-awareness system may access surgical session data that is generated during the surgical session and that is based on the one or more operations performed by the computer-assisted surgical system. Based on this surgical session data, the context-awareness system may detect an event that occurs with respect to the computer-assisted surgical system during the surgical session. The context-awareness system may then identify contextual information associated with the event and that is specific to the user role associated with the user device, and transmit, to the user device, a command for the user device to present the contextual information associated with the event.

In some examples, an additional user device may also be communicatively coupled to the computer-assisted surgical system during the surgical session. The additional user device may be associated with an additional user role that is different than the user role with which the user device is associated. The context-awareness system may accordingly abstain from directing the additional user device to present the contextual information specific to the user role. Instead, the context-awareness system may identify additional contextual information associated with the event and that is specific to the additional user role, and transmit a command to the additional user device for the additional user device to present the additional contextual information.

In additional examples, a system may include a computer-assisted surgical system that includes a manipulator arm configured to be coupled with a surgical instrument during a surgical session. The system may further include a remote computing system that is communicatively connected, by way of a network and during the surgical session, to the computer-assisted surgical system and to a user device that is communicatively paired with the computer-assisted surgical system during the surgical session. The computer-assisted surgical system may perform one or more operations with respect to a patient during the surgical session. The computer-assisted surgical system may generate, based on the one or more operations, surgical session data during the surgical session, and transmit the surgical session data to the remote computing system by way of the network. The remote computing system may identify a user profile of a user logged in to the user device. The remote computing system may receive the surgical session data generated during the surgical session from the computer-assisted surgical system by way of the network, and detect, based on the surgical session data, an event that occurs with respect to the computer-assisted surgical system during the surgical session. The remote computing system may then identify, based on the user profile of the user logged in to the user device, contextual information associated with the detected event and that is specific to the user logged in to the user device, and transmit, to the user device by way of the network, a command for the user device to present the contextual information.

To illustrate the foregoing systems, a surgical team that includes a surgeon, a nurse, and a technician (among others) may use a computer-assisted surgical system to perform a surgical procedure in which tissue is removed from a patient. The surgeon, nurse, and technician may never have worked together before as part of the same surgical team, and, as such, the nurse and technician may not be aware of certain preferences and/or tendencies of the surgeon during the surgical procedure. A context-awareness system may be configured to provide, to both the nurse and technician, contextual information based on events that occur throughout the surgical procedure so that the nurse and the technician may more effectively and efficiently assist the surgeon.

To this end, the nurse may be logged in and have access to an application running on a first user device that is communicatively paired with the computer-assisted surgical system during the surgical session. Likewise, the technician may be logged in and have access to the application running on a second user device that is communicatively paired with the computer-assisted surgical system during the surgical session. In this configuration, the first user device may be associated with a first user role that corresponds to the nurse, and the second user device may be associated with a second user role that corresponds to the technician.

During the surgical procedure, the surgeon may use master controls to manipulate dissecting forceps that are coupled to a manipulating arm of the computer-assisted surgical system. The computer-assisted surgical system may track movement of the dissecting forceps and generate surgical session data (e.g., kinematic data) representative of such movement. The context-awareness system may access this surgical session data and determine, based on the surgical session data, that a tissue removal event has occurred (i.e., that the tissue has been removed from the patient). Based on this determination, the context-awareness system may identify a first instance of contextual information associated with the tissue removal event that is specific to the user role associated with the nurse, and identify a second instance of contextual information associated with the tissue removal event that is specific to the user role associated with the technician.

For example, the first instance of contextual information may include instructions for the nurse to perform a certain nursing task that the surgeon is accustomed to having performed upon completion of the tissue removal event. The second instance of contextual information may include instructions for the technician to prepare another surgical instrument (e.g., a cautery instrument) for use by the surgeon. The context-awareness system may transmit a command to the first user device to present the first instance of contextual information to the nurse. Likewise, the context-awareness system may transmit a command to the second user device to present the second instance of contextual information to the technician.

Various benefits may be realized by the systems and methods described herein. For example, the systems and methods described herein may provide surgical team members with individually relevant contextual information in real-time during a surgical procedure, which may result in more effective and efficient collaboration and coordination among the surgical team members, and which may allow a surgeon to focus on his or her own tasks without having to individually instruct each surgical team member. Moreover, the systems and methods may predict events that may occur during the surgical session and present contextual information (e.g., advance notification) associated with such events, thus allowing surgical team members to prepare for and/or resolve such events before they occur. In some examples, the exemplary systems described herein may learn, over time, specific patterns and/or tendencies of specific surgical team members. This may allow surgical team members who have not previously worked one with another to more effectively and efficiently work as a team.

Numerous technical computing benefits may also be realized by the systems and methods described herein. For example, the systems and methods described herein may be configured to access, transform, and process data from disparate computing systems in a manner that allows the systems and methods to provide timely (e.g., real-time) information to various users by way of various computing platforms. To this end, the systems and methods described herein may seamlessly integrate with one or more special purpose computing devices to process various types of data (e.g., by applying kinematics data, image data, sensor data, and/or surgical instrument data to one or more machine learning models) in order to detect events that occur during a surgical procedure and/or identify contextual information associated with the events. In addition, the systems and methods described herein may utilize historical surgical session data generated during surgical sessions that precede a current surgical session to determine a context of the surgical session with reference to the other prior surgical sessions. In this manner, the systems and methods described herein may perform operations that are impossible to perform by a human alone. Moreover, the systems and methods described herein may improve the operation of a computer-assisted surgical system by improving efficiency, accuracy, and effectiveness of the computer-assisted surgical system.

Various embodiments will now be described in more detail with reference to the figures. The systems and methods described herein may provide one or more of the benefits mentioned above and/or various additional and/or alternative benefits that will be made apparent herein.

The systems and methods described herein may operate as part of or in conjunction with a computer-assisted surgical system. As such, an exemplary computer-assisted surgical system will now be described. The described exemplary computer-assisted surgical system is illustrative and not limiting.

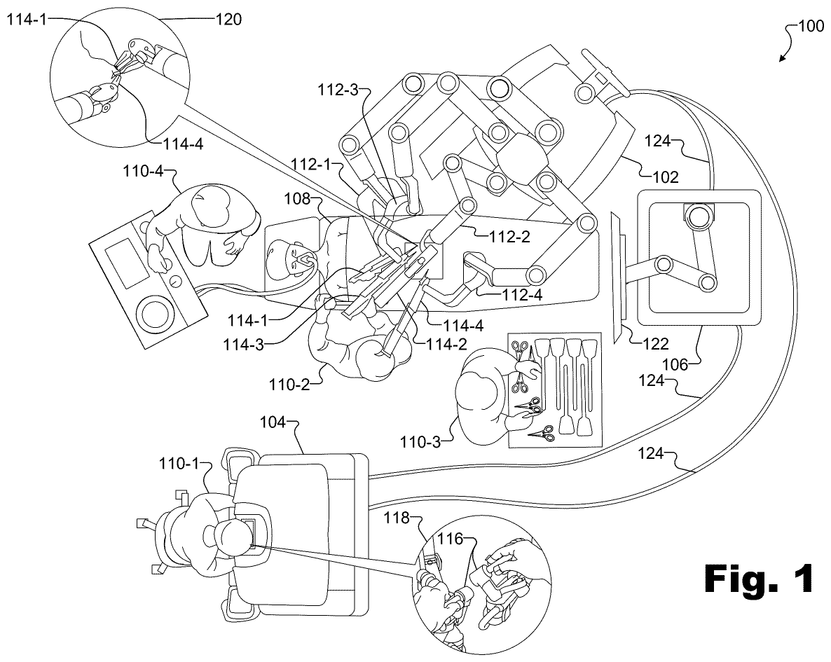

FIG. 1 illustrates an exemplary computer-assisted surgical system 100 (“surgical system 100”). As shown, surgical system 100 may include a manipulating system 102, a user control system 104, and an auxiliary system 106 communicatively coupled one to another. Surgical system 100 may be utilized by a surgical team to perform a surgical procedure on a patient 108. As shown, the surgical team may include a surgeon 110-1, a technician 110-2, a nurse 110-3, and an anesthesiologist 110-4, all of whom may be collectively referred to as “surgical team members 110.” Additional or alternative surgical team members may be present during a surgical session as may serve a particular implementation. While FIG. 1 illustrates an ongoing minimally invasive surgical procedure, it will be understood that surgical system 100 may similarly be used to perform open surgical procedures or other types of surgical procedures that may similarly benefit from the accuracy and convenience of surgical system 100. Additionally, it will be understood that the surgical session throughout which surgical system 100 may be employed may not only include an operative phase of a surgical procedure, as is illustrated in FIG. 1, but may also include preoperative, postoperative, and/or other suitable phases of the surgical procedure.

As shown, manipulating system 102 may include a plurality of manipulator arms 112 (e.g., manipulator arms 112-1 through 112-4) to which a plurality of surgical instruments 114 (e.g., surgical instruments 114-1 through 114-4) may be coupled. Each surgical instrument 114 may be implemented by any suitable surgical tool (e.g., a tool having tissue-interaction functions), medical tool, monitoring or sensing instrument (e.g., an endoscope), diagnostic instrument, or the like that may be used for a surgical procedure on patient 108 (e.g., by being at least partially inserted into patient 108 and manipulated to perform a surgical procedure on patient 108). Note that while manipulating system 102 is depicted and described herein as a cart with a plurality of manipulator arms 112 for exemplary purposes, in various other embodiments manipulating system 102 can include one or more carts, each with one or more manipulator arms 112, one or more manipulator arms 112 mounted on a separate structure within the operating room such as the operating table or the ceiling, and/or any other support structure(s). Manipulating system 102 will be described in more detail below.

Surgical instruments 114 may each be positioned at a surgical area associated with a patient. As used herein, a “surgical area” associated with a patient may, in certain examples, be entirely disposed within the patient and may include an area within the patient near where a surgical procedure is planned to be performed, is being performed, or has been performed. For example, for a minimally invasive surgical procedure being performed on tissue internal to a patient, the surgical area may include the tissue as well as space around the tissue where, for example, surgical instruments being used to perform the surgical procedure are located. In other examples, a surgical area may be at least partially disposed external to the patient. For instance, surgical system 100 may be used to perform an open surgical procedure such that part of the surgical area (e.g., tissue being operated on) is internal to the patient while another part of the surgical area (e.g., a space around the tissue where one or more surgical instruments may be disposed) is external to the patient. A surgical instrument (e.g., any of surgical instruments 114) may be referred to as being “located at” (or “located within”) a surgical area when at least a portion of the surgical instrument is disposed within the surgical area.

User control system 104 may be configured to facilitate control by surgeon 110-1 of manipulator arms 112 and surgical instruments 114. For example, user control system 104 may provide surgeon 110-1 with imagery (e.g., high-definition 3D imagery) of a surgical area associated with patient 108 as captured by an endoscope. Surgeon 110-1 may utilize the imagery to perform one or more procedures with surgical instruments 114.

To facilitate control of surgical instruments 114, user control system 104 may include a set of master controls 116 (shown in close-up view 118). Master controls 116 may be manipulated by surgeon 110-1 in order to control movement of surgical instruments 114 (e.g., by utilizing robotic and/or teleoperation technology). Master controls 116 may be configured to detect a wide variety of hand, wrist, and finger movements by surgeon 110-1. In this manner, surgeon 110-1 may intuitively perform a procedure using one or more of surgical instruments 114. For example, as depicted in close-up view 120, functional tips of surgical instruments 114-1 and 114-4 coupled to manipulator arms 112-1 and 112-4, respectively, may mimic the dexterity of the hand, wrist, and fingers of surgeon 110-1 across multiple degrees of freedom of motion in order to perform one or more surgical procedures (e.g., an incision procedure, a suturing procedure, etc.).

Although user control system 104 is depicted and described herein as a single unit for exemplary purposes, in various other embodiments user control system 104 may include a variety of discrete components, such as wired or wireless master controls 116, one or more separate display elements (e.g., a projector or head-mounted display), separate data/communications processing hardware/software, and/or any other structural or functional elements of user control system 104. User control system 104 will be described in more detail below.

Auxiliary system 106 may be configured to present visual content to surgical team members 110 who may not have access to the images provided to surgeon 110-1 at user control system 104. To this end, auxiliary system 106 may include a display monitor 122 configured to display one or more user interfaces, such as images (e.g., 2D images) of the surgical area, information associated with patient 108 and/or the surgical procedure, and/or any other visual content as may serve a particular implementation. For example, display monitor 122 may display images of the surgical area together with additional content (e.g., graphical content, contextual information, etc.) overlaid on top of or otherwise concurrently displayed with the images. In some embodiments, display monitor 122 is implemented by a touchscreen display with which surgical team members 110 may interact (e.g., by way of touch gestures) to provide user input to surgical system 100.

Manipulating system 102, user control system 104, and auxiliary system 106 may be communicatively coupled one to another in any suitable manner. For example, as shown in FIG. 1, manipulating system 102, user control system 104, and auxiliary system 106 may be communicatively coupled by way of control lines 124, which may represent any wired or wireless communication link as may serve a particular implementation. To this end, manipulating system 102, user control system 104, and auxiliary system 106 may each include one or more wired or wireless communication interfaces, such as one or more local area network interfaces, Wi-Fi network interfaces, cellular interfaces, etc.

Manipulating system 102, user control system 104, and auxiliary system 106 may each include at least one computing device configured to control, direct, and/or facilitate operations of surgical system 100. For example, user control system 104 may include a computing device configured to transmit instructions by way one or more of control lines 124 to manipulating system 102 in order to control movement of manipulator arms 112 and/or surgical instruments 114 in accordance with manipulation by surgeon 110-1 of master controls 116. In some examples, auxiliary system 106 may include one or more computing devices configured to perform primary processing operations of surgical system 100. In such configurations, the one or more computing devices included in auxiliary system 106 may control and/or coordinate operations performed by various other components (e.g., by manipulating system 102 and/or user control system 104) of surgical system 100. For example, a computing device included in user control system 104 may transmit instructions to manipulating system 102 by way of the one or more computing devices included in auxiliary system 106.

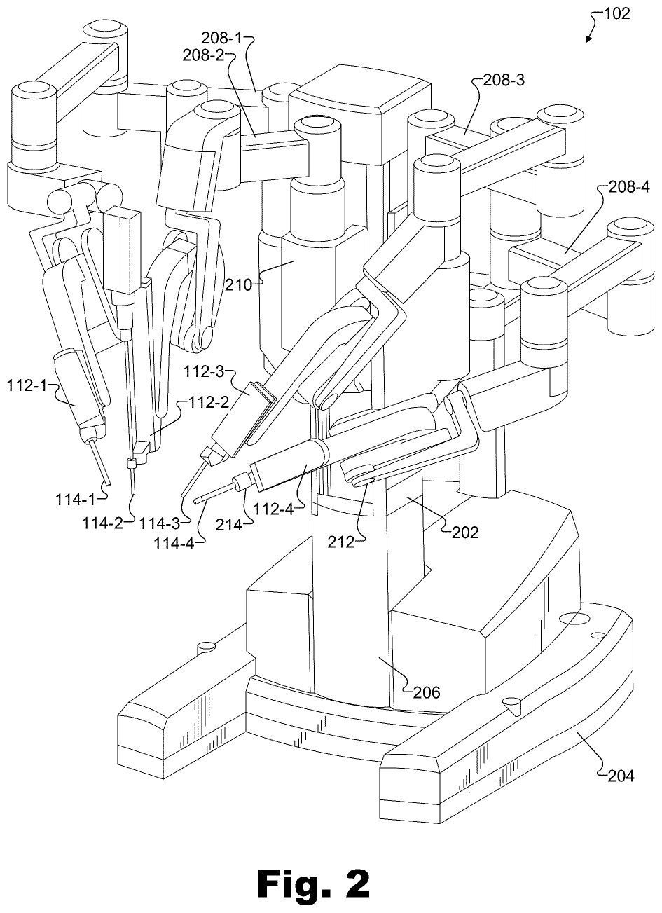

FIG. 2 illustrates a perspective view of manipulating system 102. As shown, manipulating system 102 may include a cart column 202 supported by a base 204. In some examples, cart column 202 may include a protective cover 206 that protects components of a counterbalance subsystem and a braking subsystem disposed within cart column 202 from contaminants.

Cart column 202 may support a plurality of setup arms 208 (e.g., setup arms 208-1 through 208-4) mounted thereon. Each setup arm 208 may include a plurality of links and joints that allow manual positioning of setup arms 208, and may each be connected to one of manipulator arms 112. In the example of FIG. 2, manipulating system 102 includes four setup arms 208 and four manipulator arms 112. However, it will be recognized that manipulating system 102 may include any other number of setup arms 208 and manipulator arms 112 as may serve a particular implementation.

Setup arms 208 may be manually controllable and configured to statically hold each manipulator arm 112 in a respective position desired by a person setting up or reconfiguring manipulating system 102. Setup arms 208 may be coupled to a carriage housing 210 and manually moved and situated during a preoperative, operative, or postoperative phase of a surgical session. For example, setup arms 208 may be moved and situated during a preoperative phase when surgical system 100 is being prepared and/or targeted for a surgical procedure to be performed. In contrast, manipulator arms 112 may be remotely controlled (e.g., in response to manipulation of master controls 116, as described above).

As shown, each manipulator arm 112 may have a surgical instrument 114 coupled thereto. In certain examples, three of the four manipulator arms 112 may be configured to move and/or position surgical instruments 114 that are used to manipulate patient tissue and/or other objects (e.g., suturing materials, patching materials, etc.) within the surgical area. Specifically, as shown, manipulator arms 112-1, 112-3, and 112-4 may be used, respectively, to move and/or position surgical instruments 114-1, 114-3, and 114-4. A fourth manipulator arm 112 (e.g., manipulator arm 112-2 in the example of FIG. 2) may be used to move and/or position a monitoring instrument (e.g., a stereoscopic endoscope), as will be described in more detail below.

Manipulator arms 112 may each include one or more displacement transducers, orientational sensors, and/or positional sensors (e.g., sensor 212) used to generate raw (i.e., uncorrected) kinematics information to assist in control and tracking of manipulator arms 112 and/or surgical instruments 114. For example, kinematics information generated by the transducers and the sensors in manipulating system 102 may be transmitted to an instrument tracking system of surgical system 100 (e.g., a computing device included in auxiliary system 106). Each surgical instrument 114 may similarly include a displacement transducer, a positional sensor, and/or an orientation sensor (e.g., sensor 214) in certain implementations, each of which may provide additional raw kinematics information to the tracking system to assist in control and tracking of manipulator arms 112 and/or surgical instruments 114. The instrument tracking system may process the kinematics information received from the transducers and sensors included with manipulator arms 112 and/or surgical instruments 114 to perform various operations, such as determining current positions of manipulator arms 112 and/or surgical instruments 114. Additionally, one or more surgical instruments 114 may include a marker (not explicitly shown) to assist in acquisition and tracking of surgical instruments 114 as may serve a particular implementation.

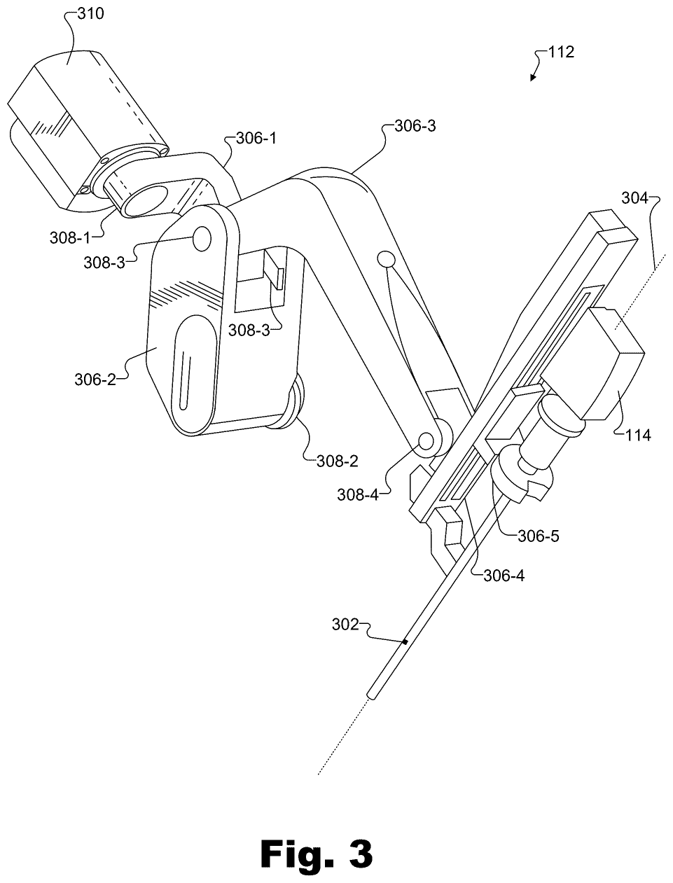

FIG. 3 illustrates a perspective view of an exemplary manipulator arm 112 (e.g., any one of manipulator arms 112-1 through 112-4). As shown, a surgical instrument 114 may be removably coupled to manipulator arm 112. In the example of FIG. 3, surgical instrument 114 is an endoscopic device (e.g., a stereo laparoscope, an arthroscope, a hysteroscope, or another type of stereoscopic or monoscopic endoscope). Alternatively, surgical instrument 114 may be a different type of imaging device (e.g., an ultrasound device, a fluoroscopy device, an MRI device, etc.), a grasping instrument (e.g., forceps), a needle driver (e.g., a device used for suturing), an energy instrument (e.g., a cautery instrument, a laser instrument, etc.), a retractor, a clip applier, a probe grasper, a cardiac stabilizer, or any other suitable instrument or tool.

In some examples, it may be desirable for manipulator arm 112 and surgical instrument 114 coupled to manipulator arm 112 to move around a single fixed center point 302 so as to constrain movement of center point 302. For example, center point 302 may be located at or near a point of insertion of a surgical instrument 114 into patient 108. In certain surgical sessions (e.g., a surgical session associated with a laparoscopic surgical procedure), for instance, center point 302 may be aligned with an incision point to the internal surgical site by a trocar or cannula at an abdominal wall. As shown, center point 302 may be located on an insertion axis 304 associated with surgical instrument 114.

Manipulator arm 112 may include a plurality of links 306 (e.g., links 306-1 through 306-5) pivotally coupled in series at a plurality of joints 308 (e.g., joints 308-1 through 308-4) near respective ends of links 306. For example, as shown, link 306-1 is pivotally coupled to a drive mount 310 at joint 308-1 near a first end of link 306-1, while being pivotally coupled to link 306-2 at joint 308-2 near a second end of link 306-1. Link 306-3 is pivotally coupled to link 306-2 near a first end of link 306-3 while being pivotally coupled to link 306-4 at joint 308-4 near a second end of link 306-3. Generally, link 306-4 may be substantially parallel to insertion axis 304 of surgical instrument 114, as shown. Link 306-5 is slidably coupled to link 306-4 to allow surgical instrument 114 to mount to and slide along link 306-5 as shown.

Manipulator arm 112 may be configured to mount to a setup arm 208 (or a joint connected thereto) by way of drive mount 310 so as to be supported and held in place by setup arm 208, as described above. Drive mount 310 may be pivotally coupled to link 306-1 and may include a first internal motor (not explicitly shown) configured to yaw manipulator arm 112 about a yaw axis of center point 302. In like manner, link 306-2 may house a second internal motor (not explicitly shown) configured to drive and pitch the linkage of manipulator arm 112 about a pitch axis of center point 302. Likewise, link 306-4 may include a third internal motor (not explicitly shown) configured to slide link 306-5 and surgical instrument 114 along insertion axis 304. Manipulator arm 112 may include a drive train system driven by one or more of these motors in order to control the pivoting of links 306 about joints 308 in any manner as may serve a particular implementation. As such, if surgical instrument 114 is to be mechanically moved, one or more of the motors coupled to the drive train may be energized to move links 306 of manipulator arm 112.

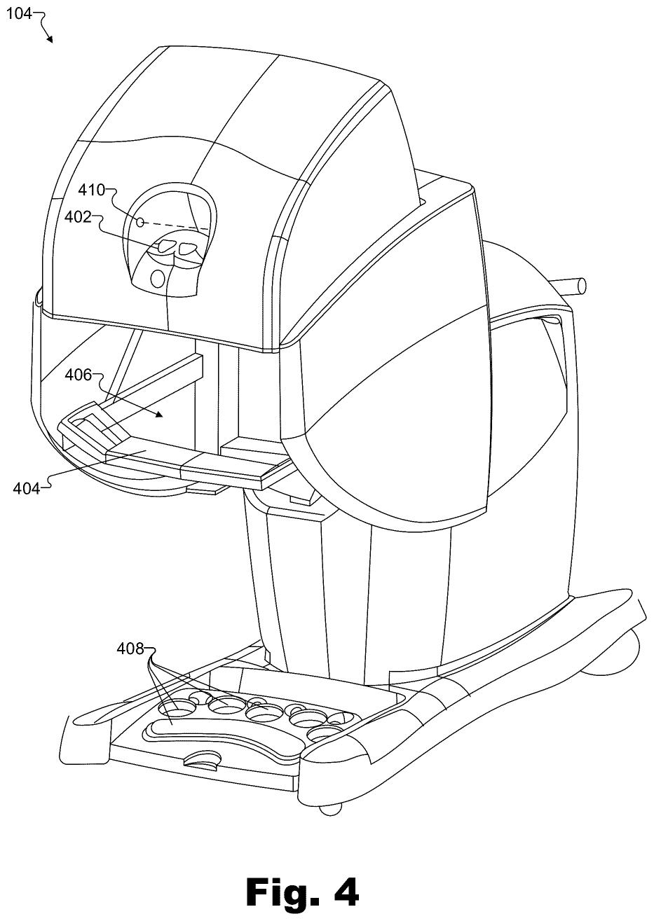

FIG. 4 illustrates a perspective view of user control system 104. As shown, user control system 104 may include a stereo viewer 402, an arm support 404, a controller workspace 406 within which master controls 116 (not shown in FIG. 4) are disposed, foot pedals 408, and a head sensor 410.

In some examples, stereo viewer 402 has two displays where stereoscopic 3D images of a surgical area associated with patient 108 and generated by a stereoscopic endoscope may be viewed by an operator (e.g., surgeon 110-1) during a surgical session. When using user control system 104, the operator may move his or her head into alignment with stereo viewer 402 to view the 3D images of the surgical area. To ensure that the operator is viewing the surgical area when controlling surgical instruments 114 of manipulating system 102, user control system 104 may use head sensor 410 disposed adjacent stereo viewer 402. Specifically, when the operator aligns his or her eyes with the binocular eye pieces of stereo viewer 402 to view a stereoscopic image of the surgical area, the operator's head may activate head sensor 410, which enables control of surgical instruments 114 by way of master controls 116. When the operator's head is removed from the area of stereo viewer 402, head sensor 410 may be automatically deactivated, which may prevent control of surgical instruments 114 by way of master controls 116. In this way, the position of surgical instruments 114 may remain static when surgical system 100 detects that an operator is not actively engaged in attempting to control surgical instruments 114.

Arm support 404 may be used to support the elbows and/or forearms of the operator while he or she manipulates master controls 116 in order to control manipulator arms 112 and/or surgical instruments 114. Additionally, the operator may use his or her feet to control foot pedals 408. Foot pedals 408 may be configured to change the configuration or operating mode of surgical system 100, to generate additional control signals used to control surgical instruments 114, to facilitate switching control from one surgical instrument 114 to another, or to perform any other suitable operation.

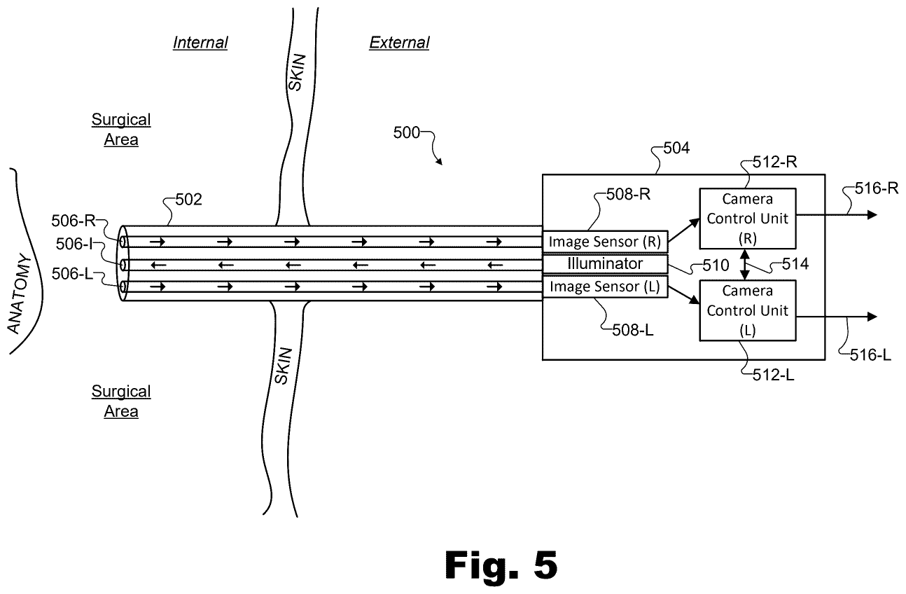

FIG. 5 illustrates an exemplary stereoscopic endoscope 500 included within surgical system 100 and located at an exemplary surgical area associated with a patient. Stereoscopic endoscope 500 may be any one of surgical instruments 114 described above.

As shown, stereoscopic endoscope 500 may include a tube 502 having a distal tip that is configured to be inserted into a patient and a camera head 504 configured to be located external to the patient. Tube 502 may be coupled at a proximal end to camera head 504 and may be rigid (as shown in FIG. 5), jointed, and/or flexible as may serve a particular implementation.

Tube 502 may include a plurality of channels 506 (e.g., a right-side imaging channel 506-R, a left-side imaging channel 506-L, and an illumination channel 506-1) configured to conduct light between the surgical area internal to the patient and camera head 504. Each channel 506 may include one or more optical fibers configured to carry light along tube 502 such that light generated within camera head 504 may be carried by illumination channel 506-I to be output at a distal end of tube 502 and, after reflecting from patient anatomy and/or other objects within the surgical area, carried by imaging channels 506-R and 506-L from the distal end of tube 502 back to camera head 504. Arrows shown within channels 506 in FIG. 5 are depicted to indicate the direction that light may travel within each channel. Additionally, tube 502 may be associated with (e.g., include) one or more lenses or other suitable optics (not explicitly shown) for focusing, diffusing, or otherwise treating light carried by channels 506 as may serve a particular implementation. In various other embodiments, there may be additional imaging and/or illumination channels. In still other embodiments, one or more image sensors and/or illuminator(s) can be positioned closer to the distal end of tube 502, thereby minimizing or even eliminating the need for imaging and/or illumination channels through tube 502.

In some examples, stereoscopic endoscope 500 may be coupled to a manipulator arm of a surgical system (e.g., one of manipulator arms 112 of surgical system 100) and positioned such that a distal tip of tube 502 is disposed within a surgical area associated with a patient. In this configuration, stereoscopic endoscope 500 may be referred to as being located at or within the surgical area, even though a portion of stereoscopic endoscope 500 (e.g., camera head 504 and a proximal portion of tube 502) may be located outside the surgical area. While stereoscopic endoscope 500 is located at the surgical area, light reflected from the surgical area may be captured by the distal tip of tube 502 and carried to camera head 504 by way of imaging channels 506-R and 506-L.

Camera head 504 may include various components configured to facilitate operation of stereoscopic endoscope 500. For example, as shown, camera head 504 may include image sensors 508 (e.g., an image sensor 508-R associated with right-side imaging channel 506-R and an image sensor 508-L associated with left-side imaging channel 506-L). Image sensors 508 may be implemented as any suitable image sensors such as charge coupled device (“CCD”) image sensors, complementary metal-oxide semiconductor (“CMOS”) image sensors, or the like. Additionally, one or more lenses or other optics may be associated with image sensors 508 (not explicitly shown). Camera head 504 may further include an illuminator 510 configured to generate light to travel from camera head 504 to the surgical area via imaging channel 506-I so as to illuminate the surgical area.

Camera head 504 may further include camera control units 512 disposed therein. Specifically, a camera control unit 512-R may be communicatively coupled to image sensor 508-R, and a camera control unit 512-L may be communicatively coupled to image sensor 508-L. Camera control units 512 may be synchronously coupled to one another by way of a communicative link 514, and may be implemented by software and/or hardware configured to control image sensors 508 so as to generate respective images 516 (i.e., an image 516-R associated with the right side and an image 516-L associated with the left side) based on light sensed by image sensors 508. As such, each respective combination of an imaging channel 506, an image sensor 508, a camera control unit 512, and associated optics may collectively be referred to as a camera included within stereoscopic endoscope 500. For example, stereoscopic endoscope 500 may include two such cameras, one for the left side and one for the right side. Such a camera may be said to capture an image 516 from a vantage point at a distal end of its respective imaging channel 506. Upon being generated by stereoscopic endoscope 500, images 516 may be displayed or otherwise processed.

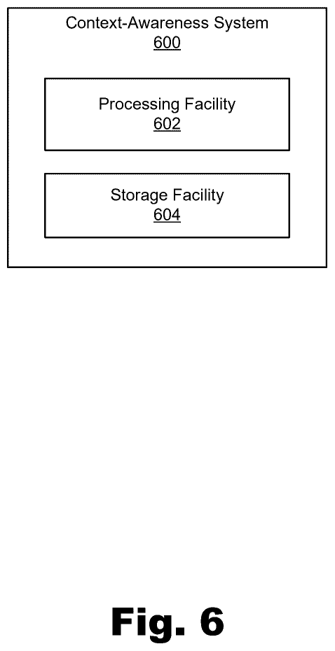

FIG. 6 illustrates an exemplary context-awareness system 600 (“system 600”) configured to provide contextual information associated with an event that occurs with respect to a computer-assisted surgical system (e.g., surgical system 100) during a surgical session. As shown, system 600 may include, without limitation, a processing facility 602 and a storage facility 604 selectively and communicatively coupled to one another. It will be recognized that although facilities 602 and 604 are shown to be separate facilities in FIG. 6, facilities 602 and 604 may be combined into fewer facilities, such as into a single facility, or divided into more facilities as may serve a particular implementation. Facilities 602 and 604 may be implemented by any suitable combination of hardware and/or software. For example, processing facility 602 may be at least partially implemented by one or more physical processors, and storage facility 604 may be at least partially implemented by one or more physical storage mediums, such as memory.

Processing facility 602 may be configured to perform various operations associated with providing contextual information associated with an event that occurs with respect to a computer-assisted surgical system. For example, processing facility 602 may determine that a user device is communicatively paired with the computer-assisted surgical system during a surgical session, identify a user role associated with the user device, access surgical session data generated during the surgical session and based on the one or more operations performed by the computer-assisted surgical system, and detect, based on the surgical session data, an event that occurs with respect to the computer-assisted surgical system during the surgical session. Processing facility 602 may be further configured to identify, based on the detected event, contextual information associated with the event and that is specific to the user role associated with the user device, and transmit, to the user device, a command for the user device to present the contextual information associated with the event. These and other operations that may be performed by processing facility 602 will be described in more detail below.

Storage facility 604 may be configured to maintain (e.g., store within a memory of a computing device that implements system 600) data generated, accessed, or otherwise used by processing facility 602. For example, storage facility 604 may be configured to maintain detection data representative of data and/or information detected or otherwise obtained by system 600, such as data representative of an identification (“ID”) of a user device, an ID of a computer-assisted surgical system, data representative of a user role associated with a user device, data representative of one or more user profiles associated with members of a surgical team, data representative of an ID of a surgical session, surgical session data, data representative of one or more events that occur during a surgical session, data representative of contextual information, etc. Storage facility 604 may be configured to maintain additional or alternative data as may serve a particular implementation.

Storage facility 604 may be configured to maintain data at any suitable location and in any suitable format or structure. For example, storage facility 604 may maintain data in one or more database formats locally (e.g., within a memory of a computing device that implements system 600) and/or remotely (e.g., within a memory of a computing device that is separate from and communicatively coupled by way of a network to system 600.

In some examples, system 600 is implemented entirely by the computer-assisted surgical system itself. For example, system 600 may be implemented by one or more computing devices included in surgical system 100 (e.g., in one or more computing devices included within manipulating system 102, user control system 104, and/or auxiliary system 106).

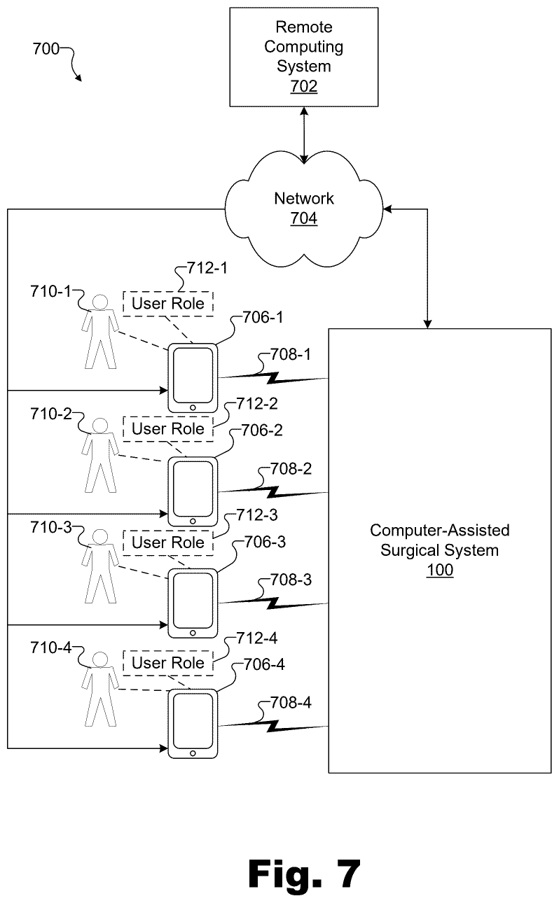

FIG. 7 illustrates an exemplary implementation 700 of system 600. In implementation 700, a remote computing system 702 may be communicatively coupled to surgical system 100 by way of a network 704. Remote computing system 702 may include one or more computing devices (e.g., servers) configured to perform any of the operations described herein. In some examples, system 600 may be entirely implemented by remote computing system 702. Alternatively, system 600 may be implemented by both remote computing system 702 and surgical system 100.

Network 704 may be a local area network, a wireless network (e.g., Wi-Fi), a wide area network, the Internet, a cellular data network, and/or any other suitable network. Data may flow between components connected to network 704 using any communication technologies, devices, media, and protocols as may serve a particular implementation.

As shown, a plurality of user devices 706 (i.e., user devices 706-1 through 706-4) may be communicatively paired with surgical system 100 by way of connections 708 (i.e., connections 708-1 through 708-4). As shown, user devices 706 may each be connected to network 704 and thereby communicate with remote computing system 702.

User devices 706 may each be any device capable of presenting contextual information to a user, whether in visual, audio, or haptic format. For example, a user device may be, but is not limited to, a mobile device (e.g., a mobile phone, a handheld device, a tablet computing device, a laptop computer, a personal computer, etc.), an audio device (e.g., a speaker, earphones, etc.), a wearable device (e.g., a smartwatch device, an activity tracker, a head-mounted display device, a virtual or augmented reality device, etc.), and/or a display device (e.g., a television, a projector, a monitor, a touch screen display device, etc.). In some embodiments, a user device may be included in surgical system 100, such as stereo viewer 402 of user control system 104 or display monitor 122 of auxiliary system 106.

As shown, a plurality of users 710 (i.e., users 710-1 through 710-4) may use or otherwise have access to user devices 706. For example, user 710-1 may use user device 706-1, user 710-2 may use user device 706-2, etc. A user (e.g., user 710-1) may have to be logged in to a user device (e.g., user device 706-1) or an application executed by the user device in order to use the user device. In some implementations, users 710 are surgical team members.

In some examples, as shown in FIG. 7, each user device 706 may be associated with a user role 712. For example, user device 706-1 may be associated with user role 712-1, user device 706-2 may be associated with user role 712-2, etc. As used herein, a “user role” may refer to a functional role or designation that a surgical team member may have during a surgical procedure. For example, a user role of “surgeon” may refer to a surgical team member tasked or trained to perform various operations that a surgeon would typically perform during a surgical procedure. Other user roles, such as “nurse”, “technician”, and “anesthesiologist” may similarly refer to different types of surgical team members tasked or trained to perform certain operations during a surgical procedure. It will be recognized that additional or alternative user roles may be specified as may serve a particular implementation. In some examples, as will be described below, system 600 may maintain data representative of a plurality of user roles that may be associated with a surgical procedure performed in connection with a computer-assisted surgical system.

A user role may be associated with a particular user device in any suitable manner. For example, user role 712-1 may be associated with user device 706-1 by specifying, within an application executed by user device 706-1, that the user role 712-1 is associated with user device 706-1. Additionally or alternatively, as will be described below, system 600 may associate a particular user role with a particular user device by maintaining data representative of the association.

Various operations that may be performed by system 600 (e.g., by processing facility 602 of system 600) and examples of these operations will now be described. It will be recognized that the operations and examples described herein are merely illustrative of the many different types of operations that may be performed by system 600.

System 600 may be configured to determine that one or more user devices (e.g., one or more of user devices 706) are communicatively paired with a computer-assisted surgical system (e.g., surgical system 100) during a surgical session. This may be performed in any suitable manner. For example, system 600 may determine that a user device is communicatively paired with the computer-assisted surgical system by determining that the user device is communicatively coupled to the computer-assisted surgical system by way of a network (e.g., network 704) and/or a direct connection (e.g., a direct wired connection and/or a direct wireless connection, such as a Bluetooth connection, a near field communication connection, etc.). Additionally or alternatively, system 600 may determine that a user device is communicatively paired with the computer-assisted surgical system by determining that the user device is logged in to a system (e.g., system 600 or any other suitable system) or a service to which the computer-assisted surgical system is also logged in, that the user device has been authenticated with the computer-assisted surgical system, that the user device is located within a predetermined physical distance of the computer-assisted surgical system (e.g., within the same room), etc. In some examples, system 600 may determine that a user device is communicatively paired with the computer-assisted surgical system by receiving (e.g., by way of a network) data from the computer-assisted surgical system and/or the user device indicating that the user device is communicatively paired with the computer-assisted surgical system.

In some embodiments, pairing of the user device with the computer-assisted surgical system may be conditioned on authentication of a user associated with the user device. For example, a pairing process may commence when the user device is detected to be connected to the same local area network as the computer-assisted surgical system, but will not be complete until the user of the user device has logged in to the user device or to an application or service provided by system 600 and accessible through the user device. Additionally or alternatively, successful pairing may further be conditioned on other parameters, such as an identity of the authenticated user matching an identity of a surgical team member previously assigned to the surgical session (e.g., at initiation or creation of the surgical session), or upon the authenticated user successfully providing user input to identify the surgical session associated with the computer-assisted surgical system with which the user device is attempting to pair (e.g., by identifying surgical session ID information, such as the patient name, etc.). System 600 may detect such successful authentication in any suitable manner (e.g., by receiving data representative of the successful authentication from the computer-assisted surgical system and/or the user device).

Once system 600 has determined that a user device is communicatively paired with a computer-assisted surgical system during a surgical session, system 600 may identify a user role associated with the paired user device. This may be performed in any suitable manner. For example, system 600 may query the user device for the user role associated with the user device. To illustrate, system 600 may transmit a request to the user device for data representative of the user role and receive, in response to the request, the data representative of the user role. System 600 may additionally or alternatively query the computer-assisted surgical system for the user role associated with the user device, in like manner. In some examples, data representative of the user role may additionally or alternatively be maintained by system 600 itself. In such configuration, system 600 may not need to query the user device or the computer-assisted surgical system to identify the user role associated with a particular paired user device.

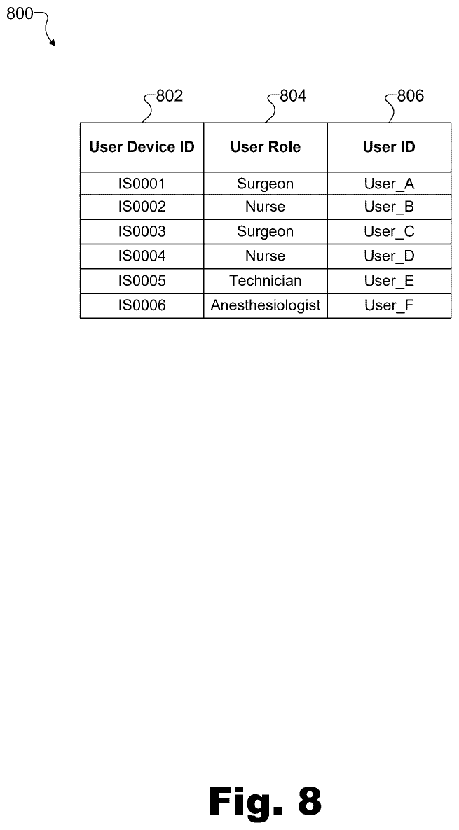

For example, FIG. 8 shows an exemplary association table 800 that may be maintained by the computer-assisted surgical system (e.g., within memory of the computer-assisted surgical system) and that may be accessed by system 600 in order to identify a user role associated with a particular user device that is communicatively paired with the computer-assisted surgical system. Association table 800 may be configured to specify which user devices are communicatively paired with the computer-assisted surgical system at any given time. For example, as shown in column 802, association table 800 may specify a plurality of user device IDs each uniquely identifying a particular user device that is communicatively paired with the computer-assisted surgical system.

Association table 800 may be further configured to specify a user role associated with each user device. For example, as shown in column 804, a user role of “surgeon” is associated with a user device that has a user device ID of “IS0001”.

Association table 800 may be further configured to specify a user ID associated with each user device that is communicatively paired with the computer-assisted surgical system. For example, as shown in column 806, a user ID of “User_A” is associated with the user device that has a user device ID of “IS0001”. The user ID may be representative of an actual user that is logged in to or otherwise using a user device or a service provided by system 600 and accessible by way of the user device.

Association table 800 may be dynamically updated as user devices are paired with or disconnected from the computer-assisted surgical system during a surgical session. For example, an additional row of data may be added to association table 800 in response to an additional user device being communicatively paired with the computer-assisted surgical system.

With system 600 being aware of which user devices are communicatively paired with the computer-assisted surgical system during the surgical session and which user roles are associated with each of the user devices, system 600 may direct the user devices to present role-specific contextual information associated with events associated with the computer-assisted surgical system and that occur during the surgical session. To this end, system 600 may access surgical session data generated during the surgical session and, based on the surgical session data, detect the events associated with the computer-assisted surgical system. Various examples of these operations will now be provided.

In some examples, surgical session data accessed by system 600 may be generated during the surgical session and may be based on or more operations performed by the computer-assisted surgical system during the surgical session. The operations performed by the computer-assisted surgical system may include any mechanical, electrical, hardware, and/or software-based operations as may serve a particular implementation. The surgical session data may be generated by the computer-assisted surgical system (e.g., by one or more components within surgical system 100), by one or more components coupled to the computer-assisted surgical system during the surgical session (e.g., one or more surgical instruments), by one or more user devices communicatively paired with the computer-assisted surgical system during the surgical session, and/or by any other device associated with the computer-assisted surgical system as may serve a particular implementation. In scenarios in which system 600 is implemented entirely by remote computing system 702, surgical session data may additionally or alternatively be generated by remote computing system 702 while, for example, remote computing system 702 tracks operations performed by the computer-assisted surgical system.

Surgical session data generated during a surgical session may include various types of data. For example, surgical session data generated during a surgical session may include kinematic data, image data, sensor data, surgical instrument data, and/or any other type of data as may serve a particular implementation.

Kinematic data may be representative of a position, a pose, and/or an orientation of a component within the computer-assisted surgical system and/or a component coupled to the computer-assisted surgical system. For example, kinematic data may be representative of a position, a pose, and/or an orientation of a manipulator arm 112 and/or a surgical instrument 114 coupled to manipulator arm 112.

Image data may be representative of one or more images captured by an imaging device coupled to the computer-assisted surgical system. For example, image data may be representative of one or more images captured by an endoscope (e.g., stereoscopic endoscope 500) coupled to a manipulator arm 112. The one or more images may constitute one or more still images and/or video captured by the imaging device. In some examples, system 600 may access image data by receiving (e.g., by way of a network) images 516 output by camera control units 512 of stereoscopic endoscope 500. In some examples, image data may additionally or alternatively include image data generated by an imaging device that is not coupled to computer-assisted surgical system 100. For example, the image data may be generated by a video camera positioned within an operating room and configured to capture video of surgical system 100, patient 108, and/or surgical team members 110.

Sensor data may include any data generated by sensors (e.g., sensors 212, 214, and/or 410) included in or associated with a computer-assisted surgical system and may be representative of any sensed parameter as may serve a particular implementation. For example, sensor data generated by sensor 410 may be indicative of whether a surgeon is actively interacting with user control system 104.

Surgical instrument data may include any data generated by a surgical

instrument (e.g., one of surgical instruments 114) and may be representative of an ID of the surgical instrument, an operational state of the surgical instrument (e.g., open, closed, electrically charged, idle, etc.), a fault code of the surgical instrument, etc.

In some examples, system 600 may additionally or alternatively access surgical session data generated by the computer-assisted surgical system during one or more other surgical sessions that, for example, precede the surgical session. For example, system 600 may generate surgical session data during a first surgical session in which the computer-assisted surgical system is used to perform a first surgical procedure with respect to a first patient. System 600 may also generate additional surgical session data during a second surgical session in which the computer-assisted surgical system is used to perform a second surgical procedure with respect to a second patient. During the second surgical session, system 600 may access both the surgical session data and the additional surgical session data. Surgical session data that is generated prior to a current surgical session may be referred to as “historical surgical session data.” As will be described below, historical surgical session data may allow system 600 to more effectively detect and/or predict an event that may occur during the second surgical session.

System 600 may additionally or alternatively access surgical session data based on operations performed by one or more computer-assisted surgical systems other than the computer-assisted surgical system being used during a particular surgical session. For example, system 600 may access surgical session data generated by a plurality of distinct computer-assisted surgical sessions located within a particular medical center, a network of hospitals, and/or any other grouping. This type of surgical data may be referred to herein as “global surgical session data” and, as will be described below, may allow system 600 to more effectively detect and/or predict an event that may occur during a particular surgical session in which a particular computer-assisted surgical system included in the grouping is used to perform a surgical procedure. In some examples, system 600 may provide an interface configured to allow a user to define a particular grouping of computer-assisted surgical systems from which surgical session data may be accessed by system 600.

System 600 may detect an event that occurs with respect to a computer-assisted surgical system during a surgical session based on surgical session data generated during the surgical session, historical surgical session data generated prior to the surgical session, and/or global surgical session data generated with respect to one or more other computer-assisted surgical systems.

An event that occurs with respect to a computer-assisted surgical system during a surgical session may include any distinct operation or action that occurs, or that may occur, with respect to the computer-assisted surgical system during the surgical session. An event may occur during a preoperative phase, an operative phase, and/or a postoperative phase of a surgical procedure.

For example, an event may include any operation or action associated with various preoperative phase operations. Such preoperative phase operations may include, but are not limited to, patient intake (e.g., admitting the patient to a medical facility, receiving patient documentation, etc.), preparing an operating room, sterilizing surgical instruments, testing the computer-assisted surgical system and equipment, draping the computer-assisted surgical system (i.e., covering one or more components of computer-assisted surgical system, such as manipulator arms 112, with a sterile or protective covering), preparing the patient for the surgical procedure (e.g., checking patient vital signs, providing intravenous fluids, administering anesthesia to the patient, bringing the patient into the operating room), and targeting the computer-assisted surgical system with respect to the patient (e.g., positioning manipulating system 102 at the patient bedside and positioning or configuring one or more manipulator arms 112).

An event may additionally or alternatively include any operation or action associated with various operative phase operations. Such operative phase operations may include, but are not limited to, opening a surgical area associated with a patient (e.g., by making an incision on external patient tissue), inserting a surgical instrument into the patient, performing surgical operations on patient tissue (e.g., by cutting tissue, repairing tissue, suturing tissue, cauterizing tissue, etc.), and closing the surgical area associated with the patient (e.g., removing surgical instruments from the patient, suturing closed the incision point, dressing any wounds, etc.).

An event may additionally or alternatively include any operation or action associated with various postoperative phase operations. Such postoperative phase operations may include, but are not limited to, removing the computer-assisted surgical system from the patient (e.g., removing manipulating system 102 from the patient bedside), patient care and recovery operations (e.g., removing the patient from the operating room, monitoring the patient as the patient recovers from the surgical procedure, etc.), cleaning the operating room, cleaning the computer-assisted surgical system and/or surgical instruments, receiving reporting documentation by surgical team members, and patient discharge operations.

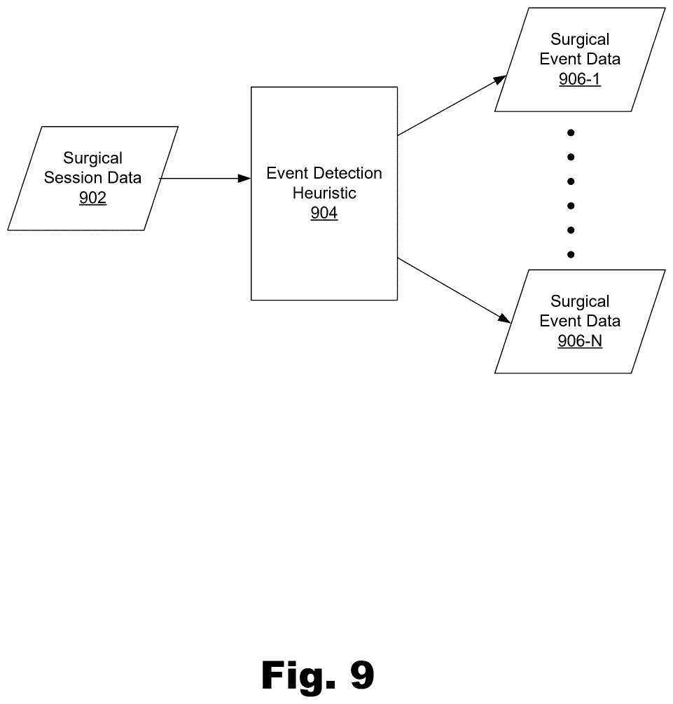

System 600 may detect an event based on surgical session data in any suitable manner. FIG. 9 shows an exemplary manner in which system 600 may detect an event based on surgical session data. As shown, system 600 may apply surgical session data 902 as an input to an event detection heuristic 904. Event detection heuristic 904 may analyze the surgical session data 902 and output various instances of surgical event data 906 (i.e., surgical event data 906-1 through surgical event data 906-N. Each instance of surgical event data 906 may represent a particular event detected by event detection heuristic 904.

Event detection heuristic 904 may include any suitable heuristic, process, and/or operation that may be performed or executed by system 600 and that may be configured detect events based on surgical session data 902. To illustrate, event detection heuristic 904 (i.e., system 600) may detect an indicator and/or pattern in surgical session data that is indicative of an occurrence of a particular event.

For example, kinematic data generated during a particular portion of a surgical session may indicate movement of a surgical instrument 114 in a suturing pattern. Additionally, surgical instrument data may indicate that the surgical instrument 114 used during the same portion of the surgical session is a needle driver. Based on this kinematic data and surgical instrument data, system 600 may determine that a suturing event is occurring, has occurred, or is about to occur.

As another example, image data representative of images 516 generated by camera control units 512 may indicate that a particular surgical instrument 114 has remained out of a view of stereoscopic endoscope 500 for a predetermined period of time. Such image data may be indicative of an idle state event (i.e., that surgical instrument 114 is an idle state).

In some examples, surgical session data 902 may include historical surgical session data, as described above. In these examples, one of the surgical event data instances 906 output by event detection heuristic 904 may be representative of an event that system 600 predicts will occur based on the historical surgical session data. For example, the historical surgical session data may include surgical session data generated during multiple surgical sessions in which the same type of surgical procedure is performed with the computer-assisted surgical system. Based on this historical surgical session data, event detection heuristic 904 may predict that a certain second event will occur following the occurrence of a certain first event.

In some examples, surgical session data 902 may include global surgical session data, as described above. In these examples, one of the surgical event data instances 906 output by event detection heuristic 904 may be representative of an event that is determined to occur based on the global surgical session data. For example, the global surgical session data may indicate that a particular kinematic data value for a particular surgical tool indicates that the surgical tool is located within a predetermined distance from patient tissue. When the actual kinematic data for the surgical tool being used during the surgical session is equal to this value, event detection heuristic 904 may detect an event that indicates that the surgical tool is actually located within the predetermined distance from patient tissue.

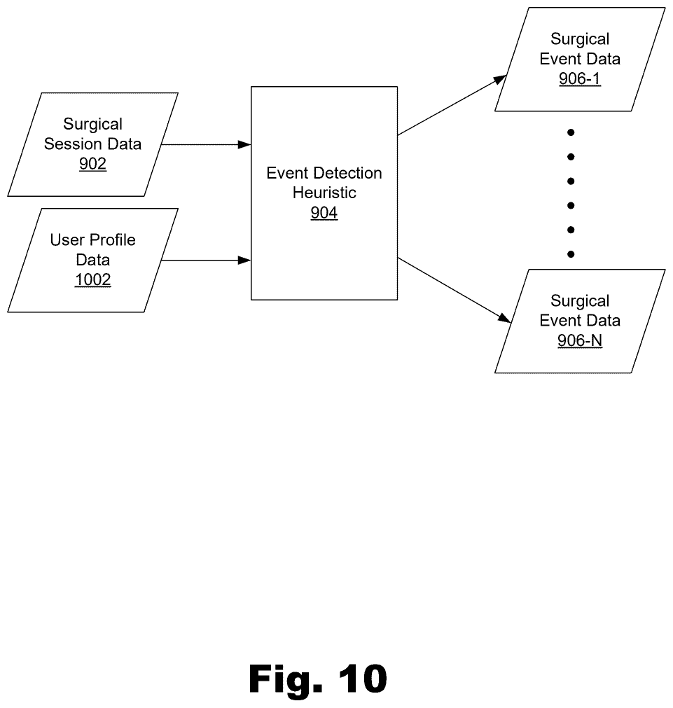

Event detection heuristic 904 may receive additional or alternative types of input as may serve a particular implementation. For example, FIG. 10 is similar to FIG. 9, but shows that event detection heuristic 904 may accept user profile data 1002 (i.e., data representative of a user profile of one or more surgical team members involved with a surgical procedure) as an additional input. In this configuration, event detection heuristic 904 may detect events based on both surgical session data 902 and user profile data 1002.

To illustrate, user profile data 1002 may include data representative of a user profile of a surgeon involved with a surgical procedure. The user profile for the surgeon, combined with the surgical session data, may indicate that the surgeon performs various operations in a certain order unique to the surgeon. Accordingly, event detection heuristic 904 may detect that a particular event is going to occur in accordance with the certain order.

In some examples, event detection heuristic 904 may implement a machine learning model. The machine learning model may use historical surgical session data and/or global surgical session data to identify one or more unique patterns of surgical system operations and associate events with the detected patterns of surgical system operations. As system 600 collects more surgical session data, surgical event data 906 output by event detection heuristic 904 may be updated or corrected as necessary. In some examples, the machine learning model may also be used to detect events and identify contextual information associated with the detected events.

When system 600 detects an event that occurs with respect to a computer-assisted surgical system during a surgical session, system 600 may identify contextual information associated with the event and that is specific to a user role associated with a user device that is communicatively paired with the computer-assisted surgical system during the surgical session. System 600 may then transmit a command to the user device for the user device to present the contextual information.

Contextual information associated with an event may include any information about the computer-assisted surgical system, the surgical session, the surgical procedure being performed during the surgical session, and/or any other information that is related to and/or provides context for the event detected by system 600. Examples of contextual information may include, without limitation, notifications (e.g., a notification that the event has occurred, is occurring, or will occur), instructions for performing an operation associated with the event (e.g., instructions for troubleshooting a detected fault, instructions for configuring various aspects of the computer-assisted surgical system), messages regarding preferences of the surgeon, etc. Contextual information may be in any format, including text, image, video, audio, and/or haptic formats.

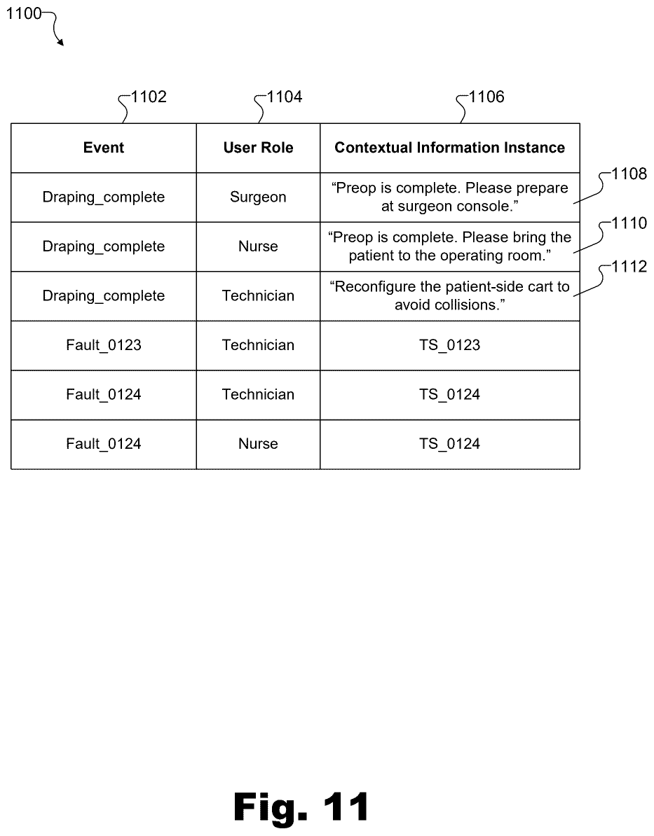

System 600 may be configured to identify contextual information associated with the detected event in any suitable way. For example, FIG. 11 shows an exemplary contextual information table 1100 that may be maintained or otherwise accessed by system 600. As shown in column 1102, table 1100 may include a plurality of entries representative of various events that may occur during a surgical session. As shown in columns 1104 and 1106, table 1100 may also list various user roles and contextual information instances associated with each event.

To illustrate, table 1100 shows that, depending on the particular user role associated with a particular user device, three different contextual information instances may be identified for a “draping_complete” event. For example, if a user device associated with a “surgeon” user role is communicatively paired with the computer-assisted surgical system during the surgical session, and the “draping_complete” event is detected, system 600 may select contextual information instance 1108 and direct the user device to present contextual information instance 1108 (e.g., in the form of a message). Likewise, if a user device associated with a “nurse” user role is communicatively paired with the computer-assisted surgical system during the surgical session, and the “draping_complete” event is detected, system 600 may select contextual information instance 1110 and direct the user device to present contextual information instance 1110 (e.g., in the form of a message). Likewise, if a user device associated with a “technician” user role is communicatively paired with the computer-assisted surgical system during the surgical session, and the “draping_complete” event is detected, system 600 may select contextual information instance 1112 and direct the user device to present contextual information instance 1112 (e.g., in the form of a message).

System 600 may also abstain from directing a user device to present a particular contextual information instance if the user device does not have a user role associated therewith that corresponds to the particular contextual information instance in table 1100. For example, system 600 may abstain from directing a user device associated with a “nurse” user role to present contextual information instances 1108 and 1112.

System 600 may generate contextual information instances based on surgical session data and surgical event data generated over time. For example, as system 600 tracks surgical system operations over time, system 600 may learn common or frequent surgical system operations performed by surgical system 100 in response to certain detected events. Utilizing historical surgical session data and surgical event data, system 600 may generate, for example, a notification or an alert of a particular type of event, and/or may generate instructions for a user to address a particular type of event.

To illustrate, system 600 may determine, from global surgical session data, that a particular configuration of manipulator arms 112 frequently results in collisions between manipulator arms 112 and/or surgical instruments 114. Accordingly, system 600 may generate an alert to be presented by way of a user device associated with a particular user role when the particular configuration of manipulator arms 112 is detected. As another example, system 600 may determine, from historical surgical session data, that a grasping-type surgical instrument is frequently unable to be removed from a patient because the grasp has not been released. Accordingly, system 600 may generate a notification for a surgeon to release the grasp of the surgical instrument prior to removal, and a notification for the technician to wait to remove the surgical instrument until the surgeon has released the grasp (which event may also be alerted to the technician).

Additionally or alternatively, system 600 may generate contextual information instances based on user input, such as user input provided by way of user devices. In some examples, the user input may be provided in real time during the surgical session. For example, a technician may be unable to remove a forceps instrument from the patient because it is currently grasping tissue. The technician may provide a message to the surgeon to release the grip of the forceps. The message may be provided through the user device associated with the technician (e.g., by way of a textual message, a voice input, or a pre-selected message), or the message may be provided verbally and detected by a microphone located within the operating room. System 600 may store the message as a contextual information instance and use it in the future when the same event (i.e., a failure to remove a forceps instrument) is detected.

Additionally or alternatively, user input of a contextual information instance may be provided after the operative phase of the surgical procedure or after the surgical session. For instance, during the postoperative phase of the surgical procedure, the surgeon or another user may review a log of events detected during the surgical session and select or provide contextual information associated with one or more of the detected events. System 600 may store the contextual information and use it in the future when the same or similar events are detected.

Additionally or alternatively, system 600 may customize contextual information based on a user profile of a surgical team member. For instance, a first surgeon may prefer certain type of instrument for a particular procedure, while a second surgeon may prefer a different type of instrument for the same procedure. Accordingly, contextual information associated with an event (e.g., commencement of a tissue cutting event) data may include first contextual information based on a first user (e.g., a notification specific for a technician to prepare a cautery instrument preferred by a first surgeon) and contextual information based on a second user (e.g., a notification specific for a technician to prepare dissecting forceps preferred by a second surgeon).

Additionally or alternatively, prior to identifying and/or selecting contextual information, system 600 may access user profile data to determine one or more user specific parameters to use in selecting the contextual information. User specific parameters may include any information associated with the user, and may include, without limitation, a training level of the user, an experience level of the user (e.g., the number of surgical procedures in which the user has participated), a history of detected events associated with the user, frequency of usage of particular surgical instruments by the user, frequency of occurrence of detected faults associated with the user, timing information of the user (e.g., the amount of time the user takes to accomplish certain operations), and the like. For instance, when a system fault has been detected with respect to a surgical instrument, contextual information facility may identify the contextual information to be presented to a technician based on a training level of the technician. For a technician that has received minimal training with respect to addressing the fault, video instructions explaining how to resolve the fault may be identified as the contextual information to be presented to the technician. On the other hand, for a technician that has received in-depth training with respect to addressing the fault and has successfully resolved the fault several times previously, a simple notification that the fault has been detected may be identified as the contextual information to be presented to the technician.

Once system 600 has identified contextual information associated with an event and that is specific to a user role associated with the user device, system 600 may direct the user device to present the contextual information. In this manner, a user of the user device may be presented with the contextual information. System 600 may direct the user device to present the contextual information in any suitable manner. For example, system 600 may transmit, to the user device, a command for the user device to present the contextual information.

If the contextual information is stored locally at the user device, the command transmitted to the user device from system 600 may direct the user device to present the contextual information by accessing the locally stored contextual information. If the contextual information is not stored locally at the user device, system 600 may also transmit, or cause to be transmitted, data representative of the identified contextual information along with the command. For example, data representative of the identified contextual information may be stored at a remote computing device (e.g., a remote server) different than system 600. In this scenario, system 600 may be configured to direct the computing device to transmit data representative of the contextual information to the user device. In yet another embodiment, the command transmitted to the user device by system 600 may direct the user device to access (e.g., request and receive) the contextual information from a remote computing device that maintains the contextual information.

A user device may present contextual information associated with an event in any suitable manner. For example, the user device may display the contextual information by way of a display screen in the form of a message, a graphic, an image, a video, and/or any other suitable visual content. In some examples, the contextual information may be displayed within a graphical user interface associated with an application executed by the user device and provided by or otherwise associated with system 600.

Additionally or alternatively, a user device may present contextual information by presenting audio content representative of the contextual information. The audio content may, in some instances, include an audible spoken message, an audible alarm or other sound, etc.

Additionally or alternatively, a user device may present contextual information by presenting haptic content representative of the contextual information. The haptic content may, for example, include a vibration indicative of a notification received by the user device.

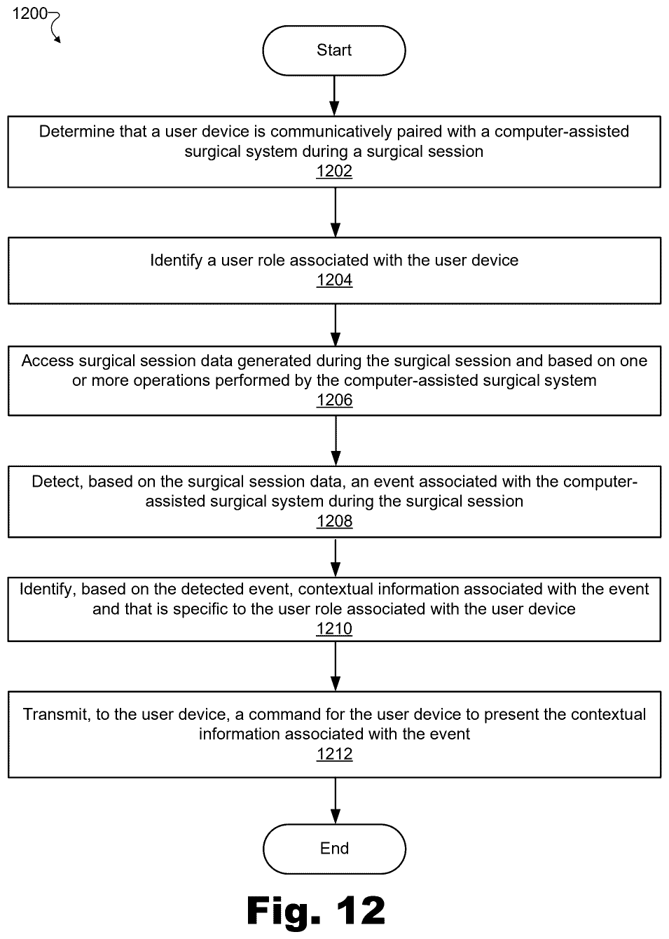

FIG. 12 shows an exemplary context-awareness method 1200. While FIG. 12 illustrates exemplary operations according to one embodiment, other embodiments may omit, add to, reorder, combine, and/or modify any of the steps shown in FIG. 12. One or more of the operations shown in in FIG. 12 may be performed by system 600, any components included therein, and/or any implementation thereof.

In operation 1202, a context-awareness system determines that a user device is communicatively paired with a computer-assisted surgical system during a surgical session in which the computer-assisted surgical system performs one or more operations with respect to a patient. Operation 1202 may be performed in any of the ways described herein.

In operation 1204, the context-awareness system identifies a user role associated with the user device. Operation 1204 may be performed in any of the ways described herein.

In step 1206, the context-awareness system accesses surgical session data generated during the surgical session and based on the one or more operations performed by the computer-assisted surgical system. Operation 1206 may be performed in any of the ways described herein.

In step 1208, the context-awareness system detects, based on the surgical session data, an event that occurs with respect to the computer-assisted surgical system during the surgical session. Operation 1208 may be performed in any of the ways described herein.

In step 1210, the context-awareness system identifies, based on the detected event, contextual information associated with the event and that is specific to the user role associated with the user device. Operation 1210 may be performed in any of the ways described herein.

In step 1212, the context-awareness system transmits, to the user device, a command for the user device to present the contextual information associated with the event. Operation 1212 may be performed in any of the ways described herein.

In certain embodiments, one or more of the systems, components, and/or processes described herein may be implemented and/or performed by one or more appropriately configured computing devices. To this end, one or more of the systems and/or components described above may include or be implemented by any computer hardware and/or computer-implemented instructions (e.g., software) embodied on at least one non-transitory computer-readable medium configured to perform one or more of the processes described herein. In particular, system components may be implemented on one physical computing device or may be implemented on more than one physical computing device. Accordingly, system components may include any number of computing devices, and may employ any of a number of computer operating systems.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

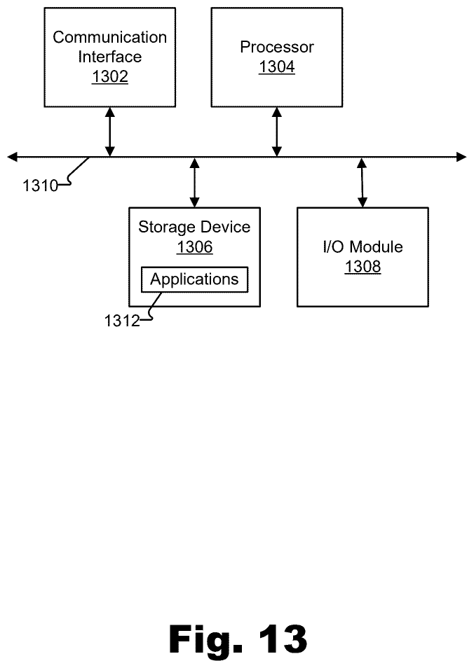

FIG. 13 illustrates an exemplary computing device 1300 that may be specifically configured to perform one or more of the processes described herein. As shown in FIG. 13, computing device 1300 may include a communication interface 1302, a processor 1304, a storage device 1306, and an input/output (“I/O”) module 1308 communicatively connected via a communication infrastructure 1310. While an exemplary computing device 1300 is shown in FIG. 13, the components illustrated in FIG. 13 are not intended to be limiting. Additional or alternative components may be used in other embodiments. Components of computing device 1300 shown in FIG. 13 will now be described in additional detail.

Communication interface 1302 may be configured to communicate with one or more computing devices. Examples of communication interface 1302 include, without limitation, a wired network interface (such as a network interface card), a wireless network interface (such as a wireless network interface card), a modem, an audio/video connection, and any other suitable interface.

Processor 1304 generally represents any type or form of processing unit capable of processing data or interpreting, executing, and/or directing execution of one or more of the instructions, processes, and/or operations described herein. Processor 1304 may direct execution of operations in accordance with one or more applications 1312 or other computer-executable instructions such as may be stored in storage device 1306 or another computer-readable medium.

Storage device 1306 may include one or more data storage media, devices, or configurations and may employ any type, form, and combination of data storage media and/or device. For example, storage device 1306 may include, but is not limited to, a hard drive, network drive, flash drive, magnetic disc, optical disc, RAM, dynamic RAM, other non-volatile and/or volatile data storage units, or a combination or sub-combination thereof. Electronic data, including data described herein, may be temporarily and/or permanently stored in storage device 1306. For example, data representative of one or more executable applications 1312 configured to direct processor 1304 to perform any of the operations described herein may be stored within storage device 1306. In some examples, data may be arranged in one or more databases residing within storage device 1306.

I/O module 1308 may include one or more I/O modules configured to receive user input and provide user output. One or more I/O modules may be used to receive input for a single virtual reality experience. I/O module 1308 may include any hardware, firmware, software, or combination thereof supportive of input and output capabilities. For example, I/O module 1308 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1308 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing device 1300. For example, one or more applications 1312 residing within storage device 1306 may be configured to direct processor 1304 to perform one or more processes or functions associated with processing facility 602 of system 600. Likewise, storage facility 604 of system 600 may be implemented by storage device 1306 or a component thereof.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
at least one physical computing device communicatively coupled to a computer-assisted surgical system during a surgical session in which the computer-assisted surgical system performs one or more operations with respect to a patient;
wherein the at least one physical computing device is configured to:
determine that a user device used by a first surgical team member is communicatively paired with the computer-assisted surgical system during the surgical session,
identify a user role associated with the user device,
access surgical session data generated during the surgical session and based on the one or more operations performed by the computer-assisted surgical system,
detect, based on the surgical session data, an event that occurs with respect to the computer-assisted surgical system during the surgical session,
Identify, based on the detected event and based on a user profile of a second surgical team member different from the first surgical team member, contextual information associated with the event and that is specific to the user role associated with the user device, and
transmit, to the user device, a command for the user device to present the contextual information associated with the event.
